(19) United States
(12) Patent Application Publication
(10) Pub. No.: US 2018/0036099 A1
(43) Pub. Date: Feb. 8, 2018

(54) LIQUID SEALING COMPOUND AND USE THEREOF IN ENDODONTIC PROCEDURES

(71) Applicants: Ghassan YARED, Toronto (CA); Gustavo De-Deus, Rio de Janerio (BR)

(72) Inventors: Ghassan YARED, Toronto (CA); Gustavo De-Deus, Rio de Janerio (BR)

(21) Appl. No.: 15/670,666

(22) Filed: Aug. 7, 2017

(30) Foreign Application Priority Data
Aug. 8, 2016 (CA) 2,943,435

(51) Int. Cl. A61C 5/40; A61K 6/00; A61C 5/50

(52) U.S. Cl. CPC A61C 5/40; A61C 5/50; A61K 6/0038

(57) ABSTRACT

Provided herein is a curable sealer for use in sealing a canal wall in a root canal treatment or retreatment procedure, including an adhesive compound having a cytotoxicity sufficiently low as to be suitable for use in the root canal procedure; and a flowability of at least 46 mm at 23° Celsius. Provided herein are uses of the sealer for sealing a canal wall. Also provided is a curable sealer for use in sealing a canal wall in a root canal treatment or retreatment procedure, including an adhesive compound having: a cytotoxicity sufficiently low as to be suitable for use in the root canal procedure; and a flowability at 23° Celsius about equal to the flowability of water. Provided herein are uses of the sealer for sealing a canal wall.

### CROSS-REFERENCE TO RELATED APPLICATION

This application claims priority to Canadian Patent Application No. 2,943,435 filed on Aug. 8, 2016, the contents of which are incorporated by reference herein.

### TECHNICAL FIELD

This invention relates to sealers used in endodontic procedures. In particular, this invention relates to a high flowability sealer for use in a root canal treatment or retreatment procedure.

### BACKGROUND

An important endodontic procedure, known as a “root canal” procedure, involves removing organic material from the root canals of an infected tooth and filling the canal with an inert obturating material such as gutta-percha gum.

An effective root canal procedure avoids extraction of the infected tooth. In this procedure, a dentist or endodontist utilizes a series of endodontic instruments, for example files, for the debridement, cleaning and sterilization of the root canal. These files are rotated within the canal to clean the canal surfaces, removing debridement (organic) material in the process, facilitating improved irrigation, and in some cases shaping the canal for easier filling with the obturating material.

Once the pulp has been removed from the root canal, a smear layer remains. The smear layer is potentially infected, and its removal allows more efficient penetration of intracanal medications into the dentinal tubules and a better interface between the filling material and the root canal walls. A final flush with chelating agents and antiseptic irrigating solutions is needed to remove the smear layer, following which the canal must be filled as soon as possible to avoid decomposition of the exposed dentin and potentially further infection.

Filling the prepared canal involves applying a sealer to the canal wall, to close off pores and microtubules in the dentin surface, and compression of an inert obturating compound such as gutta-percha gum into the canal. Known sealers have a low flowability, which reduces extrusion through the apical opening at the bottom of the canal. Apical extrusion of the sealing material from the distal end of the canal into the jaw bone can result in treatment failure or other complications.

Because the sealer has a relatively low flowability, it does not inherently intrude into the pores and microtubules in the dentin surface or completely fill the gap between the obturating filler and the canal wall, which is required in order to prevent infection. Therefore, an effective filling procedure requires that the gutta-percha obturating compound be heated to a semi-fluid state to make it malleable enough to fill the entire canal, and compressed into the canal after the sealer has been applied to the canal wall in order to ensure that the sealer is in turn compressed against the canal wall over the entire surface area of the canal wall. This requires that the canal be large enough to permit manipulation of the heated gutta-percha in the canal so that it intrudes into the hard-to-reach areas of the canal and pushes the sealer into every gap between the obturating filler (for example heated gutta-percha) and the canal wall, and into the hard-to-reach areas of the canal. However, there is exhaustive evidence that gaps are frequently observed following the filling of the root canal and that many hard-to-reach areas in the canals remain unfilled.

A conservative approach, which is always recommended in root canal treatment and re-treatment applications, recognizes that it is advantageous to limit the root canal to the smallest possible size while removing all infected tissue. This maintains as much of the tooth material as possible. However, a smaller canal space makes it even more difficult and potentially impossible to compress the heated gutta percha sufficiently to ensure that the process of condensing (compacting) the obturating material will fully compress the viscous sealer against the canal wall, over the entire surface area of the canal wall, so as to fill pores and dentinal tubules to the maximum extent possible. In result, in many cases the dental professional must remove more of the tooth material than is strictly required to cure the tooth of infection, in order to ensure that the subsequent filling step will succeed in filling the canal while minimizing air gaps.

Current modern root canal techniques do not allow the filling of many canals that are minimally enlarged or canals that have been cleaned but not enlarged. However, the removal of additional material from the tooth solely to facilitate the subsequent filling of the canal reduces the structural integrity of the tooth, increasing the risk of fracture both during and after the treatment procedure.

It would accordingly be beneficial to be able to use a sealer that has a sufficiently high flowability, or in other words a sufficiently low viscosity, as to seal the canal wall solely under the influence of fluid dynamics, without requiring compressive pressure. This would allow the pre-filling root canal shaping procedure to be significantly more conservative, requiring the removal of less material, and thus retain the integrity of the tooth structure as much as possible while allowing the canal to be properly sealed and filled. In many cases this would also avoid the need for the expensive equipment used in conventional root canal filling techniques.

A sealer that has a sufficiently low viscosity (high flowability) would allow the filling of canals that are cleaned, but without requiring any additional root canal shaping solely to promote effective sealer coverage through compaction of the obturating filler. However, requirement for using a high viscosity sealer in order to avoid the risk of apical extrusion is widely known and well documented.

For example, in ORSTAVIK (“Physical properties of root canal sealers: measurement of flow, working time, and compressive strength”, International Endodontic Journal, 1983, pp. 99-107, Volume 16, Blackwell Scientific Publications) at page 105 Orstavik teaches “ . . . an optimal flow or flow range should be established, defining a consistency sufficiently thin to permit flow into instrumented root canals, yet sufficiently thick to avoid inadvertent flow of the material into the periapical tissues.”.

Similarly, in NEGM et al. (“A study of the Viscosity and Working Time of Resin-based Root Canal Sealers, Journal of Endodontics, October 1985, pp 442-445, Volume 11 Number 11”), at page 442 Negm et al. teach: “The viscosity must be great enough to ensure movement of the material along the canal only when operative forces of appropriate magnitude are used, otherwise the distribution of the material would be unduly influenced by extraneous and relatively uncontrollable influences such as gravity and surface tension (10).”

More recently, in TANOMARU-FILHO et al. (“Radiopacity and flow of different endodontic sealers”, Acta Odontol. Latinoam, 2013, pp 121-125, Volume 26 No. 2) at pages 121 and 122, Tanomaru-Filho et al. teach “Endodontic sealers should be capable of penetrating accessory canals and irregularities of the root canal system. However, excessive flow may increase the risk of material extrusion beyond the apex, which can promote damage to the periodontal tissues.” (at pp. 121-122).

In COLLARES et al. (“Influence of radiopaque fillers on physicochemical properties of a model epoxy resin-based root canal sealer”, Journal of Applied Oral Science, 2013, pp 533-539, Volume 21(6)) at page 537 Collares et al. teach “In addition, the flow of the sealer cannot be too high due to a possible periapical extrusion, which could compromise apical healing and lead to decreased tooth longevity. The film thickness of experimental sealers was similar to widely used commercial sealers, which have a film thickness of approximately 50 μm.”

Flowability is inversely related to viscosity, and is defined as the capacity to move by flow that characterizes fluids and loose particulate solids. The flowability of a sealer can be measured by placing a specific amount of sealer between two glass plates and then placing a weight on the top plate for a specific amount of time. The distance the sealer spreads out, measured in millimetres, determines the flowability of the sealer according to the ANSI/ADA's specification No. 57 or ISO/DIS 6876:2010.

For example, a volume of 0.5 mL of a popular sealer mixed according to the manufacturer's recommendations was placed on a glass plate. At 180 seconds (±5 seconds) after the commencement of mixing, the second 100-gram glass plate was placed on top of the mixed sealer, followed by a 20-gram weight for a total mass of 120 grams. Ten minutes after the start of mixing, the weight was removed and the value of the diameter of the compressed disc of sealer was measured (see CHANG et al., “Comparison of the rheological properties of four root canal sealers”, International Journal of Oral Science, 2014, pp 56-61, Volume 7, Nature). The highest flowability value determined with this test according to the ANSI/ADA' s specification No. 57 and reported in articles published in scientific journals is 45 mm.

Thus, according to conventional teachings, procedures and commercially available sealers, there is an upper limit to the flowability of a sealer that will function in a root canal treatment to seal the pores and microtubules in the dentin wall of the canal, albeit under the compressive influence of the dental practitioner's gutta-percha manipulation, and additionally plug the apical opening with minimal risk of the sealer extruding through to the jawbone or surrounding tissue.

Some authors go as far as teaching that the sealer should be tacky when mixed (see for example CHHABRA et al., “Fate of Extruded Sealer: A Matter of Concern”, Journal of Oral Health and Community Dentistry, September 2011, pp 168-172, Volume 5(3), wwwjohcd.org), which requires a very high viscosity with the attendant need to heat the obturating compound and compact and condense it so as to minimize the incidence of gaps, to ensure full coverage and proper adhesion of the sealer in the canal.

United States Patent No. 8,044,113 issued Oct. 25, 2011 to Dentsply International, Inc., which is incorporated herein by reference in its entirety, refers to the need for a root canal sealing compound to have a low viscosity in order to enter into dentin canals in the root canal, and teaches a compound having a viscosity of less than 100 Pa·s, preferably in the range from 1 to 80 Pa·s and more preferably in the range from 1 to 20 Paas. However, even the lower limit of the sealer viscosity referenced in this patent is significantly more viscous than water (approximately 0.93 mPa·s) and oils (between 33 and 400 mPa·s), and thus substantial pressure must be used in compacting the obturating filler in order to mechanically force the sealer into the pores and microtubules within the canal wall, given irregularities in the canal space leading to hard-to-reach areas. This requires that the root canal be enlarged more than is strictly necessary to cure the tooth pathology, with the attendant disadvantages mentioned herein, such as the documented risk of sealer extruding through the enlarged root canal to the jawbone or surrounding tissue.

### SUMMARY

In accordance with an aspect, there is provided a curable sealer for use in sealing a canal wall in a root canal treatment or retreatment procedure, comprising an adhesive compound having: a cytotoxicity sufficiently low as to be suitable for use in the root canal procedure; and a flowability of at least 46 mm at 23° Celsius.

In an embodiment, the flowability is in the range of 46 mm to about equal to the flowability of water. In an embodiment, the flowability is from 46 mm to about 49 mm. In an embodiment, the flowability is from 46 mm to about 56 mm. In an embodiment, the flowability is about 130 mm. In an embodiment, the flowability is above 130 mm.

In accordance with an aspect, there is provided a use of the sealer for sealing a canal wall in a root canal treatment or retreatment procedure in preparation for filling by an obturating filler.

In accordance with another aspect, there is provided a use of the sealer for filling a root canal in a root canal treatment or retreatment procedure without any obturating filler.

The sealer may comprise a compound diluted to the desired flowability within the range, including a natural or synthetic bioadhesive.

According to another aspect, there is provided a curable sealer for use in sealing a canal wall in a root canal treatment or retreatment procedure, comprising an adhesive compound having a cytotoxicity sufficiently low as to be suitable for use in the root canal procedure; and a flowability at 23° Celsius about equal to the flowability of water.

### BRIEF DESCRIPTION OF THE DRAWINGS

In drawings which illustrate by way of example only a preferred embodiment of the invention:

FIG. 1 is a side elevational cross-section of a tooth requiring a root canal procedure, prior to canal preparation;

FIG. 2 is a side elevational cross-section of the tooth of FIG. 1 showing the canal following conventional preparation for filling with enlargement and shaping;

FIG. 3 a side elevational view of an obturating cone suitable for filling the canal space after cleaning;

FIG. 4 is a side elevational cross-section of the tooth of FIG. 1 showing obturating filling material implanted in the cleaned root canal space without shaping or enlargement; and

FIG. 5 is a side elevational cross-section of the tooth of FIG. 1 showing a sealer according to the invention deposited in the cleaned root canal space without shaping or enlargement.

### DETAILED DESCRIPTION

The present invention provides a sealer for use in a root canal treatment or retreatment procedure having a flowability in a range of 46 mm and above, for example to about the flowability of water at 23° Celsius. Such a sealer has a sufficiently high flowability (low viscosity) as to meet the requirements for a successful root canal treatment or retreatment procedure without requiring compaction of the obturating filler in order to ensure full coverage and proper adhesion of the sealer, the elimination of all gaps and voids, and the filling of irregularities and hard-to-reach areas.

A sealer according to the invention thus may provide one or more of a number of advantages. The sealer will flow into cleaned canal spaces without any compaction procedure or other application of pressure, thereby promoting the simplest and safest way to fill a root canal. Modern canal cleaning techniques (such as Sonendo and PIPS) have been developed to clean and disinfect the canal without any enlargement to avoid weakening the tooth, which could result in its eventual fracture and lead to extraction. However, these modern canal cleaning techniques do not allow the filling of the canals using the current conventional filling techniques unless the canals are first shaped/enlarged.

Where a sealer exhibits anti-bacterial properties, the higher flowability may further the anti-bacterial effect by being able to reach more areas thereby to kill more bacteria (SIQUEIRA et al., “Antimicrobial Activity and Flow Rate of Newer and Established Root Canal Sealers”, Journal of Endodontics, May 2000, pp 274-277, Volume 25 Number 5). It has been shown that sealers with the greatest flowability have the highest anti-bacterial activity (NAWAL et al., “A comparative evaluation of antimicrobial efficacy and flow properties for Epiphany, Guttaflow and AH-Plus sealer”, International Endodontic Journal, 2011, pp 1-4, Volume 44).

A sealer according to the invention provides a solution for the filling of canals and their hard-to-reach areas/irregularities that have been cleaned and disinfected, but without any enlargement or with only a minimal enlargement (at which a conventional filling technique will not allow the filling of the hard-to-reach areas/irregularities. The sealer will flow into the hard-to-reach/irregularities of a canal that has been cleaned (pulp tissue removed) and not shaped or minimally shaped. In some cases this may also avoid the need for expensive equipment used in conventional root canal filling techniques. A sealer according to the invention also has reduced cytotoxicity because it is less concentrated than conventional root canal sealers, has a longer working time before setting which increases opportunities for the sealer to migrate into hard to reach portions/irregularities of the cleaned canal, and has less contraction (dimensional changes leading to the formation of micro-gaps) of the sealer upon setting (hardening).

To achieve this result, the sealer should have a flowability above 46 mm and preferably about equal to the flowability of water at 23° Celsius. In some cases the sealer can be used without requiring an obturating filler, particularly a sealer having a flowability in the upper portion of the range of about 46 mm to about that of water. In other cases, where the sealer is in the lower portion of this flowability range, an obturating filler such as a gutta-percha cone may be gently inserted into the root canal after it has been filled with sealer, without any pumping action, to assist in maximizing the coverage of the sealer and its intrusion into microtubules, pores and irregularities in the surface of the canal wall, and the canal irregularities. Other means to assist in maximizing the coverage of the sealer are mechanical (vibration) or ultrasonic energy.

Examples of available compounds which can be adapted to have a characteristic flowability range above or equal to 46 mm at 23° Celsius and the necessary adhesion and resistance to degradation are:
AH+™ by Dentsply™ and Pulp Canal Sealer™ by Kerr Endodontics™. These materials have been used successfully in canals conventionally dried with paper points. It is noted however, that canals cannot be completely dried with paper points or otherwise; the canal will always present some humidity or wetness—in humid conditions. These two commercially available compounds have moderate humidity resistance, which makes them suitable for a root canal filling where some degree of humidity is always present.
Amalgabond™ (2-Hydroxyethyl methacrylate) by Parkell™. This material has also been used successfully in contact with vital pulp tissues in very humid conditions and has excellent adhesive properties in a wet environment.
Bioadhesives, for example those produced by mussels or mussels-inspired synthetic adhesives. These adhesives are particularly suitable for use in the environment of a root canal, considering the wet environments in which the adhesives are used by mussels.

For the referenced commercially-available sealers the proportions of the components are varied from the recommended mixing instructions. For example, in the case of AH+ the base and catalyst components are mixed outside of the recommended proportions until the desired flowability (viscosity) has been reached. Similarly for the Kerr Pulp Canal sealer the liquid and power ratio can be changed to produce a more fluid composition having the desired flowability (viscosity). This can be tested using the glass plate flowability test referenced above, which is readily available to the practitioner, or by any other suitable means including viscosity testing. Alternatively, sealing compounds may be created by the manufacturer having a flowability falling within the range of the invention.

Once the canal has been cleaned, or minimally prepared and cleaned, the high-flowability sealer is dispensed from a syringe or other metering dispenser at the top of the canal, or within the canal if care is taken not to pressurize the sealer as it is deposited. The sealer will flow into the canal, filling it completely including the hard-to-reach areas and irregularities. In the process the walls of the canal are coated with the sealer.

When a fluid sealer according to the invention is placed in the canal, the likelihood of apical extrusion is dependent on the amount of the sealer (i.e. the weight of the sealer that will carry the sealer forward into the canal); the surface of the canal, since friction from the rough canal surface will oppose the forward movement deeper into the canal; the positive pressure from tissue fluids around the canal opening, which will counter the weight of the liquid; and tissue present at the opening of the canal, which can form a physical barrier that will prevent the extrusion of the liquid. Within the flowability range according to the invention, as long as no substantial pressure is applied the sealer will not extrude because the weight of the volume of sealer required in a small canal space is not sufficient to overcome the resistance opposing the flow of sealer by these other parameters. Thus, using a sealer having the characteristic flowability range of the invention causes a plug to form at the apical opening as the sealer sets, preventing extrusion from the canal, as long as the sealer is introduced into the canal without applying any substantial pressure.

In some embodiments an obturating filler (e.g. gutta-percha gum) is inserted into the canal, at room temperature, to fill the canal space. If using a gutta-percha cone the cone will typically displace some sealer from the canal, so a thin obturating cone may be used to reduce the amount of displacement, without compaction of the cone or other application of pressure.

The insertion of a thin gutta-percha cone will not cause extrusion unless the cone is applied using a pumping action. Thus, where a gutta-percha cone filler is used it should not be compressed; it should be inserted into the canal in a very passive manner without any pumping action to avoid pressurizing the uncured sealer. The tooth can then be capped or filled in the conventional manner, for example by applying a crown.

In other embodiments an obturating cone may not be needed for some canals that have been cleaned but not shaped (enlarged) where a liquid sealer having a very high flowability, the closer to the flowability of water the better, is used to fill the entire cleaned canal space and allowed to set. In some cases this may be the only way to fill cleaned canals that have been cleaned but not shaped without the risk of extrusion. In this technique the sealer performs the functions of both sealing the canal wall and providing a permanent filling—a plug—in the canal. The setting time is somewhat longer than where an obturating filler such as gutta-percha is used, but the procedure is considerably simpler. X-rays can be used to determine if there are voids or gaps in the filling, however except in extreme cases it is typically assumed that all voids and spaces have been filled.

### EXAMPLE 1

A suitable high-flowability (low-viscosity) sealer was prepared by mixing the components of the widely-used sealer AH+™ by Dentsply™ outside of the directed proportions, to produce a composition within the flowability range according to the invention. AH+ is described in U.S. Pat. No. 5,624,976 to Klee, which is incorporated herein by reference. AH+ samples were prepared by mixing the components at different ratios, in each case to obtain a more fluid state than that directed by the manufacturer, i.e. to increase their flowabilities to greater than or equal to 46 mm and up through a range of flowabilities above 46 mm to about the flowability of water. The samples were prepared and tested as described below.

### EXAMPLE 2

A sealer according to the invention was prepared from a widely-used sealer (Pulp Canal Sealer by Kerr). The sealer components, a powder and a liquid (as purchased), when mixed according to the mixing directions, produce a composition having a flowability of about 36 mm (see below). By mixing a higher proportion of the liquid component with the powder than directed by the manufacturer, a series of samples each having a different flowability at or above 46 mm to approach or equal the flowability of water, was prepared and tested as described below. It will be noted that, in tests that were done, the width of the 100-gram glass slabs used were a limiting factor in determining precise flowability values at the higher ranges. In particular, the limited size of the glass slabs used limited reliable sample measurement of acceptable flowabilities to 130 mm. Tested samples having 130 mm flowabilities, as well as those that exceeded 130 mm to approach or equal the flowability of, for example, water, were found to be usable for high-flow sealing without extrusion.

Pulp Canal Sealer by Kerr calls for a mixing ratio of 1 unit powder:1 unit liquid. This sealer was used for experimentation because flowability and viscosity values for the recommended mix ratio are available in the product literature and it is easy to control the mixing ratio. The following mix ratios were attempted to create a liquid sealer from Pulp Canal Sealer by Kerr with higher flowability:

a) 1 unit powder:1unit liquid—The flowability results were about 36 mm, as tested using the ANSI/ADA specification referred to above.

b) 1 unit powder:1.25 units liquid—The flowability results (38 mm) were very similar to the recommended mixing ratio of 1 unit powder:1 unit liquid under a 120-g weight (a 100-g glass plate with a 20-g weight atop the glass plate). There was no apparent increase in flowability of the resulting mixture. The resulting sealer mixed in this ratio barely penetrated in an artificial empty (and not shaped) canal in a resin block, and was unable to penetrate deeper in the canal when the resin block containing the artificial canal was vibrated for a long duration of time. Similarly, the resulting sealer mixed in this ratio barely penetrated in an artificial empty and minimally shaped canal in a resin block, and was unable to penetrate deeper in the canal when the resin block containing the artificial canal was vibrated for a long duration of time or when a thin gutta-percha cone (obturating filler) was inserted passively, without any pumping action.

c) 1 unit powder:1.5 units liquid—In this case there was an increased flowability compared to a) and b); range between 46 mm and 49 mm) under a 120-g weight. The resulting sealer mixed in this ratio filled an artificial empty canal in a resin block to almost ¼-½ of its length, and was able to fill the full length of the canal when the resin block containing the artificial canal was vibrated. Surprisingly, and contrary to the general understanding in the art that the lower value of 45 mm flowability achievable using AH+ as directed by the literature is somewhat of a gold standard for maximal flowability without the problem of extrusion, the 46 mm to 49 mm flowability sealer according to the present invention presented superior filling and did not in fact extrude from the tip of the canal in the absence of opposing forces from surrounding tissues and positive pressure from tissue fluids.

d) 1 unit powder:1.75 units of liquid—The increase in flowability was significant compared to b) and c). The sealer flowed to approximately 56 mm under a 120-g weight. The resulting sealer mixed in this ratio filled an artificial empty canal in a resin block to more than one half of its length, and was able to fill the full length of the canal when the resin block containing the artificial canal was vibrated for a shorter period of time than in the previous test c). Advantageously, despite the significant increase in flowability, the sealer did not actually extrude from the tip of the canal in the absence of opposing forces from surrounding tissues and positive pressure from tissue fluids.

e) 1 unit powder:2 (two) units of liquid—In this case there was an increase in flowability resulting in flow to approximately 62mm under a 120-g weight. When placed at the orifice of an artificial empty canal the artificial canal was filled nearly completely without any extrusion.

f) 1 unit powder:21 (twenty one) units of liquid—In this case there was significant increase in flowability resulting in flow to approximately 130 mm under a 120-g weight. When placed at the orifice of an artificial empty canal the artificial canal was filled completely without any extrusion.

It is expected that, given particularly the results for d) and e) above, ratios of powder to liquid that are between 1:2 and 1:21, such as 1:3, 1:4, 1:5 and so forth would work to completely or at least nearly completely fill a canal without extrusion as described above. Furthermore, it has been observed that flowabilities that exceed 130 mm to approach or equal the flowability of, for example, water, were found to be similarly usable for high-flow sealing without extrusion.

In both Example 1 and Example 2 the mixed sealer was deposited in the mesial (coronal) third of canals in extracted teeth and artificial canals using a syringe, slowly so as not to pressurize the sealer, to fill the canal. All canals were only minimally shaped and did not permit the effective use of conventional filling techniques. In some canals, a thin gutta-percha cone was inserted passively, without any pumping action. In other canals the sealer was left to set undisturbed. The apex of the canals was observed with a dental microscope to confirm or rule out the presence of apical extrusion. Extrusion did not occur with any of the sealer samples in the absence of opposing forces from surrounding tissues and positive pressure from tissue fluids.

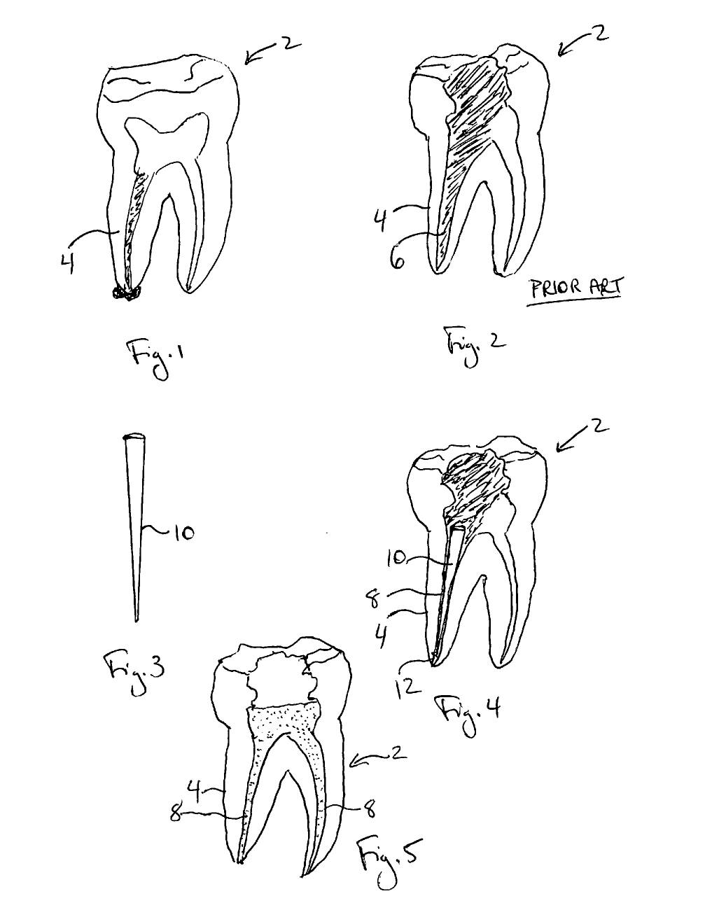

FIG. 1 illustrates a tooth 2 having an infected root 4. To save the tooth 2 the root canals must be cleaned, i.e. cleared of organic debris and sterilized, in preparation for filling. FIG. 2 illustrates the root canal 6 after cleaning according to conventional techniques, whereby the root canal 6 has been shaped and enlarged by an instrument (not shown), for example a hand-held file or motorized tool actuating a reciprocating file. This allows sufficient room for compaction of an obturating filler, for example a gutta-percha cone, in order to pressurize the sealer and force it into pores, microtubules and irregularities in the canal wall.

FIG. 4 illustrates the same tooth 2 following filling of the root canal 8 by an obturating filler, for example the thin gutta-percha cone 10 illustrated in FIG. 3, in accordance with the invention. The root canal 8 in this procedure has not been shaped or enlarged, but has merely been cleared of organic debris and sterilized. The high flowability sealer poured into the canal 8 before insertion of the obturating cone 10 actually formed a plug 12 at the apex of the canal 8, preventing apical extrusion as the obturating cone 10 was carefully inserted to avoid pressurizing the sealer during the filling step. There is significantly more tooth material of the root 4 surrounding the root canal 8 in the tooth that underwent the root canal treatment according to the invention. The other roots of the tooth 2 would be treated similarly.

FIG. 5 illustrates the same tooth 2 following filling of the root canals 8 by a sealer in accordance with the invention. The root canals 8 in this procedure have not been shaped or enlarged, but have merely been cleared of organic debris and sterilized. Again, the high flowability sealer poured into the canals 8 actually formed a plug 12 at the apex of the canal 8, preventing apical extrusion. In this embodiment there is significantly more tooth material of the root 4 surrounding the root canals 8 in the tooth that underwent the root canal treatment according to the invention than that illustrated in FIG. 2 where enlargement/shaping was required to complete the sealing step.

1. A curable sealer for use in sealing a canal wall in a root canal treatment or retreatment procedure, comprising an adhesive compound having:
a cytotoxicity sufficiently low as to be suitable for use in the root canal procedure; and
a flowability of at least 46 mm at 23° Celsius.

2. The sealer of claim 1 comprising a bioadhesive.

3. Use of the sealer of claim 1 for sealing a canal wall in a root canal treatment or retreatment procedure in preparation for filling by an obturating filler.

4. Use of the sealer of claim 1 for filling a root canal in a root canal treatment or retreatment procedure without any obturating filler.

5. The curable sealer of claim 1, wherein the flowability is in the range of 46 mm to about 130 mm.

6. The curable sealer of claim 5, wherein the flowability is from 46 mm to about 49 mm.

7. The curable sealer of claim 5, wherein the flowability is from 46 mm to about 56 mm.

8. The curable sealer of claim 5, wherein the flowability is about 130 mm.

9. The curable sealer of claim 1, wherein the flowability is above 130 mm.

10. A curable sealer for use in sealing a canal wall in a root canal treatment or retreatment procedure, comprising an adhesive compound having:
a cytotoxicity sufficiently low as to be suitable for use in the root canal procedure; and
a flowability at 23° Celsius about equal to the flowability of water.

11. The sealer of claim 10 comprising a bioadhesive.

12. Use of the sealer of claim 10 for sealing a canal wall in a root canal treatment or retreatment procedure in preparation for filling by an obturating filler.

13. Use of the sealer of claim 10 for filling a root canal in a root canal treatment or retreatment procedure without any obturating filler.
